(19) United States
(12) Patent Application Publication
(10) Pub. No.: US 2012/0330178 A1
(43) Pub. Date: Dec. 27, 2012

(54) METHOD AND APPARATUS FOR MULTIMODAL MOBILE SCREENING TO QUANTITATIVELY DETECT BRAIN FUNCTION IMPAIRMENT

(75) Inventors: Reuben H. Kraft, Aberdeen, MD (US); Ashley E. Eidsmore, Havre de Grace, MD (US); Jessica R. Compton, Gunpowder, MD (US)

(73) Assignee: U.S. Government as represented by the Secretary of the Army, Adelphi, MD (US)

(21) Appl. No.: 13/530,391

(22) Filed: Jun. 22, 2012

Related U.S. Application Data
(60) Provisional application No. 61/500,992, filed on Jun. 24, 2011.

(51) Int. Cl. A61B 5/0484; A61B 3/113; A61B 5/11

(52) U.S. Cl. 600/544

(57) ABSTRACT

A method and apparatus including a mobile device for administering and quantitatively determining a Risk Index that a user has suffered an impairment of brain function.

### CROSS-REFERENCE TO RELATED APPLICATIONS

This application claims priority to U.S. Provisional Patent Application Ser. No. 61/500,992 filed Jun. 24, 2011, which is incorporated by reference herein in its entirety.

### GOVERNMENT INTEREST

Governmental Interest—The invention described herein may be manufactured, used and licensed by or for the U.S. Government.

### BACKGROUND

1. Field

Embodiments of the present invention generally relate to detecting impairment of brain function, and more particularly, to a method and apparatus for multimodal mobile screening to quantitatively detect brain function impairment, such as a mild traumatic brain injury.

2. Description of the Related Art

Detecting impairment of brain function, such as a mild Traumatic Brain Injury (mTBI) is a growing medical concern, not only for the United States military, but also to the public in general due to its long-term effects on the brain. MTBI caused by blast or impact, is one of the most common combat wounds suffered by service members, and sports related head injuries, including concussions, are also a topic of great concern for both the players and organizations, such as the National Football League (NFL). According to the Defense and Veterans Brain Injury Center over 200,000 service members have sustained a TBI since 2000. Others have estimated that up to 28 percent of service members deployed to Iraq or Afghanistan have sustained at least one mTBI. A mTBI, also referred to as acute military concussion, leads to persistent post-concussion symptoms and often remains undiagnosed. Some common symptoms of mTBI include: loss or decreased level of consciousness, loss of memory before or after injury, alteration in mental state at the time of the injury, neurological deficits (loss of balance, change in vision, etc.), and/or intracranial lesions.

Diagnosis of MTBI is difficult and continues to be an area of significant research within the brain injury scientific community. Currently, there is no “gold standard” test for diagnosis of mild brain impairment, such as mTBI. However, there are promising screening techniques that probe various functional areas of the brain, for example quantitative electroencephalography (QEEG) testing that examines brain synchronization and postural stability testing that examines vestibular function. Despite some promising results, these techniques lack a significant degree of accuracy for both sensitivity and specificity of mild impairment, such as mTBI. Such techniques, unfortunately, are prone to noise and system instability. Additionally, these techniques are not well suited for rapid deployment and use by untrained personnel, and particularly in a combat area or at a sporting event.

Therefore, there is a need in the art for an improved method and apparatus for detecting impairment of brain function in a quantitative way with both accuracy and specificity.

### SUMMARY OF THE INVENTION

Embodiments of the present invention relate to an apparatus comprising a mobile device for administering and quantitatively determining a Risk Index of a user suffering an impairment of brain function. In one embodiment the Risk Index is defined as a function: f(QEEG, cognitive assessment test 1, cognitive assessment test N, where N=2 or more).

Other embodiments of the present invention include one or more methods for administering in a mobile manner a quantitative determination of a user suffering an impairment of brain function, by performance of a multimodal screening procedure and subsequent calculation of a Risk Index, substantially as shown in and/or described in connection with at least one of the figures and as set forth more completely in the claims.

Various advantages, aspects and features of the present disclosure, as well as details of an illustrated embodiment thereof, will be more fully understood from the following description and drawings

### BRIEF DESCRIPTION OF THE DRAWINGS

While the method and apparatus described herein is by way of example for several embodiments and illustrative drawings, those skilled in the art will recognize that the method and apparatus for administering in a mobile manner a quantitative determination of a user suffering an impairment of brain function, by performance of a multimodal screening procedure and subsequent calculation of a Risk Index, is not limited to the embodiments or drawings described. It should be understood, that the drawings and detailed description thereto are not intended to limit embodiments to the particular form disclosed. Rather, the intention is to cover all modifications, equivalents and alternatives to those embodiments falling within the spirit and scope of the method and apparatus for administering in a mobile manner a quantitative determination of a user suffering an impairment of brain function by performance of a multimodal screening procedure and subsequent calculation of a Risk Index, as defined by the appended claims. Any headings used herein are for organizational purposes only and are not meant to limit the scope of the description or the claims.

The word “may” is used in a permissive sense (i.e., meaning having the potential to), rather than the mandatory sense (i.e., meaning must), and the words “include”, “including”, and “includes” mean including, but not limited to.

Furthermore, throughout the drawings, like reference numerals will be understood to refer to like parts, components and structures.

FIG. 1 depicts a block diagram of an apparatus useful for multimodal mobile screening to detect mild traumatic brain injury in accordance with one or more embodiments of the invention;

FIG. 2 depicts a startup screen of a smart phone embodiment of the apparatus of FIG. 1, showing the option to start a new brain function impairment screening test or create a new baseline.

FIG. 3 depicts a flow diagram of a method for multimodal mobile screening to detect brain function impairment according to one or more embodiments of the invention, using the apparatus of FIG. 1.

FIG. 4 depicts a flow diagram of a QEEG/Flanker Arrow task invoked by the method of FIG. 3.

FIGS. 5 and 6 depict screen displays using the apparatus of FIG. 1 to show cue and imperative stimulus for the Flanker Arrow task as performed by the method of FIG. 4.

FIG. 7 depicts a flow diagram of a Paired Associate Learning and memory task as invoked by the method of FIG. 3.

FIGS. 8, 9 and 10 depict screen displays using the apparatus of FIG. 1 to show shape stimulus for the Paired Associate Learning and memory task as performed by the method of FIG. 7.

FIG. 11 depicts a flow diagram of a balance task as invoked by the method of FIG. 3.

FIG. 12 depicts a screen display using the apparatus of FIG. 1 to show the instructions for the balance task as performed by the method of FIG. 11.

FIG. 13 depicts typical EEG results collected during a brain function impairment screening test conducted using the apparatus of FIG. 1.

FIG. 14 depicts scoring criteria for the QEEG/Flanker Arrow task of FIG. 4.

FIG. 15 depicts scoring criteria for the Paired Associate Learning memory task of FIG. 7.

FIG. 16 depicts a simple user screen showing the risk of having brain function impairment that is calculated from results of the multimodal mobile screening method of FIG. 3.

FIG. 17 depicts a result summary screen illustrating how the user scored on each of the individual tasks of the method of FIG. 3.

FIG. 18 depicts the raw data summary screen illustrating options to display the raw data acquired by each of the individual tasks of the method of FIG. 3, and also gives the user the option to send the raw data to another via email.

FIGS. 19 and 20 each depict a screen display using the apparatus of FIG. 1 to show the instructions and tests for the optional eye tracking test.

### DETAILED DESCRIPTION OF THE INVENTION

Embodiments of the present invention comprise a method and apparatus to use multimodal mobile screening for detecting brain function impairment, such as mild traumatic brain injury (mBTI) or the like. A portable diagnostic device, which in one embodiment may comprise a smart phone of convention design, serves as a computing platform and hardware interface for implementing a mobile application that can screen a user of the portable device in a quantitative manner, for determining a risk index that is indicative of the user having suffered a brain function impairment. More specifically, modules typically found in smart phones, such as a touch screen, accelerometers and a Bluetooth short-range transceiver, are selectively accessed by the mobile application so as to lead the user of the smart phone through a battery of neurocognitive tasks, so as to provide a multimodal screening tool for determining a risk index of brain function impairment. The Bluetooth module, in conjunction with an Electroencephalogram (EEG) brainwave sensor/transmitter can use wireless EEG technology to monitor brain waves of the user while the user is being led through one or more of the plurality of screening tasks. In one embodiment, three neurocognitive tasks are presented to the user while user performance data is acquired and stored by the portable diagnostic device. A first task is a Flanker Arrow task which evaluates reaction time, and when coupled with EEG monitoring of the users brain waves, provides a means to measure event related potentials and make a quantitative evaluation of the users' performance of this task. A second task is a Paired Associate Learning (PAL) task which is designed to evaluate memory, and a third task is a postural stability (i.e., balancing) task. These three specific tasks are chosen in this embodiment of a multimodal screening tool because they are orthogonal, that is they task three different areas of the brain, thereby leading to a more robust determination of brain function. After presentation of the three tasks, the portable diagnostic device processes data acquired during user performance of the tasks so as to provide a display to the user of a quantitative risk index indicative of the user having a brain function impairment. The portable diagnostic device also provides the option for the user to view a summary or the raw data relating to the performance of each of the tasks, as well as the option to use the communication module of the portable diagnostic device to transmit the acquired data and/or the determined risk index to another location.

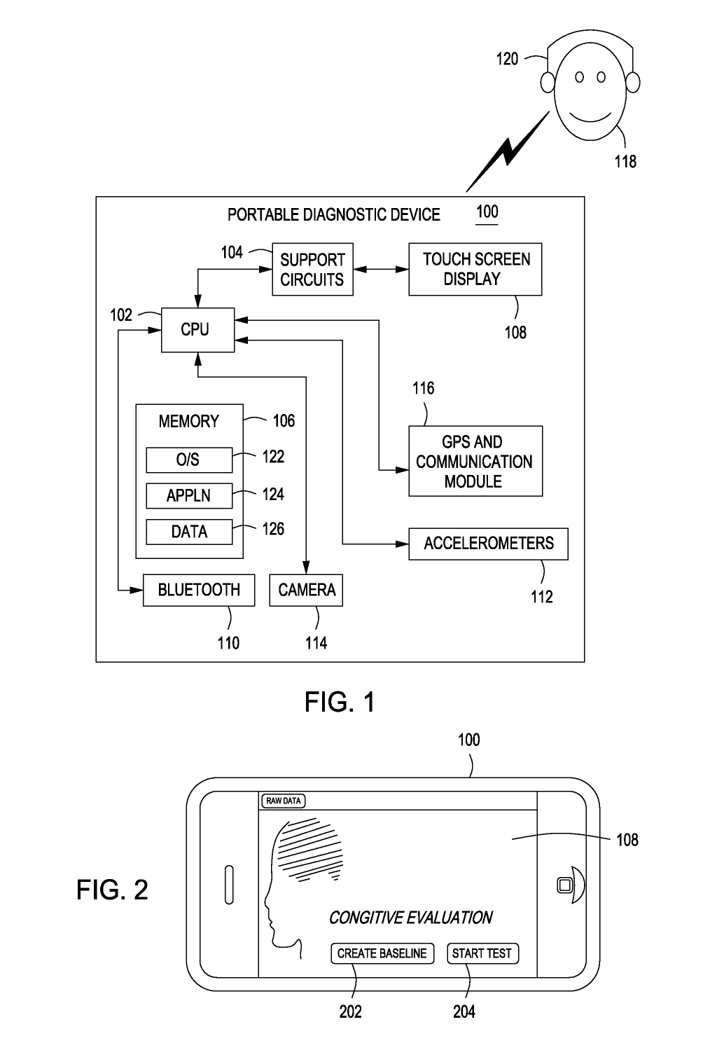

FIG. 1 is a block diagram of a portable diagnostic device 100 in accordance with one or more embodiments of the invention. In one embodiment, the apparatus 100 comprises a smart mobile phone, such as an iPhone®, having a housing that includes a central processing unit (CPU) 102, support circuits 104, a memory 106, a touch screen display 108, a wireless Bluetooth transceiver 110, accelerometers 112, a camera 114 and a GPS and cellular communication module 116. In one embodiment, a user 118 of the portable diagnostic device 100 places on his head a wireless EEG sensor/transceiver 120 for acquiring brain wave signals from the user during the performance of neurocognitive tasks, for transmission to the portable diagnostic device 100. A NeuroSky MindSet headset, available from NeuroSky, Inc. located in San Jose, Calif. 95113, can be used as the EEG sensor/transceiver 120 to capture the EEG signals. In some embodiments the EEG signals may be transmitted to the portable diagnostic device 100 via a wired connection, in which event a wireless Bluetooth transceiver would not be required in the portable diagnostic device 100. Additionally, in some embodiments the camera 114, which may be used to perform an eye tracking task, may not be included in the diagnostic device 100, and therefore in some embodiments performance of an eye tracking task would not form a part of the multimodal screening process. Although a smart phone, such as an APPLE iPhone® is shown in one illustrated embodiment as the portable diagnostic device, the portable diagnostic device may be of any form sufficient to provide the functions described herein, such as a dedicated device intended solely for this purpose, and may or may not comprises one or both of long or short range mobile communications circuitry, a touch screen display or accelerometers.

The CPU 102 comprises one or more commercially available microprocessors or microcontrollers that facilitate data processing and storage, as well as mobile communications. The various support circuits 104 facilitate the operation of the CPU 102 and include one or more clock circuits, power supplies, cache, input/output circuits, displays, and the like. The memory 106 comprises at least one of a Read Only Memory (ROM), Random Access Memory (RAM), disk drive storage, optical storage, removable storage and/or the like. The memory 106 comprises an operating system (OS) 122 for controlling the overall operation of the smart phone, an application 124 and a data storage area 126. According to some embodiments of the invention, the operating system 122 generally manages various resources of the smart phone (e.g., the touch screen display 108, the GPS and communication module 116, the Bluetooth communication module 110, the accelerometers 112 and the camera 114) so that apparatus 100 generally operates as a smart phone, however upon CPU 102 accessing memory 106 so as to execute application 124, the smart phone will serve as a computing platform and hardware interface to provide a multimodal screening tool for determining a risk index of brain function impairment, such as a mild traumatic brain injury, of a user of the smart phone. Smart phones typically include the above noted central processing unit (CPU) 102, support circuits 104, memory 106, touch screen display 108, wireless Bluetooth transceiver 110, accelerometers 112, a camera 114 and a GPS and cellular communication module 116, and such components are well known to those of ordinary skill in this technology. Accordingly, further description of these components is not provided, unless specifically needed to complete the description of one of the illustrated embodiments.

FIG. 2 depicts a startup screen of a smart phone embodiment of the apparatus of FIG. 1, showing the option to start a new-screening test for brain function impairment or create a new baseline. More specifically, upon execution of application 124, touch screen display 108 displays to the user of the diagnostic device the choice to touch one of two buttons, a “create baseline” button 202 or a “start test” button 204. The ability to allow the user to run the neurocognitive assessment tasks in a baseline mode is an effective way to help increase accuracy of the quantitative determination of risk index, by providing a history of test results for a given user that can be used as a baseline comparison for subsequent running of the neurocognitive assessment tasks. Although the ability of the multimodal screening application 124 to provide for a baseline mode is shown herein, such feature is not specifically required in all embodiments of the invention, and a quantitative risk index can be calculated without the requirement of having previously acquired baseline data.

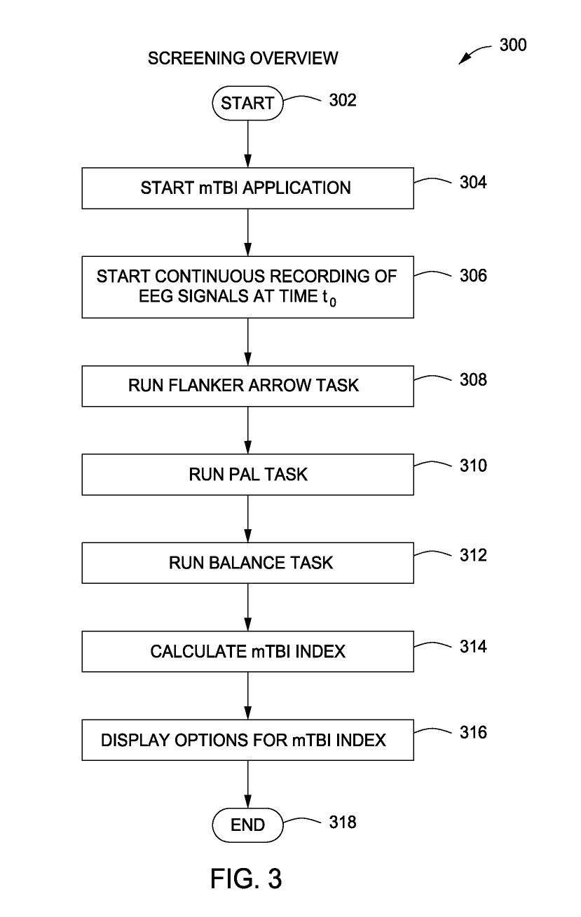

FIG. 3 depicts a flow diagram of a method 300 for multimodal mobile screening to detect brain impairment according to one or more embodiments of the invention, using the apparatus of FIG. 1. The method 300 is invoked when application 124 is executed by CPU 102.

The method 300 starts at step 302 and proceeds to step 304. At step 304, application 124 initializes apparatus 100 so as to start operation as a portable diagnostic device. At step 306, the data portion 126 of memory 106 begins to continuously record, at time t₀, the EEG signals applied to apparatus 100 from the wireless EEG sensor/transceiver 120 mounted on the head of a user 118. At step 308, application 124 presents a Flanker Arrow task to the user and records the users' performance data in data portion 126 of memory 106. At step 310, application 124 presents a Paired Associate Learning (PAL) task to the user and records the users' performance data in data portion 126 of memory 106. At step 312, application 124 presents a Postural Stability (balance) task to the user and records the user's performance data in data portion 126 of memory 106. At step 314, apparatus 100 processes the acquired and stored performance data so as to calculate a risk index that the user 120 has a brain function impairment. In particular, if baseline performance data for the user is available, the risk index will indicate if the user 120 has suffered a brain function impairment subsequent to the acquiring of the performance data during the baseline mode. Although in the illustrated embodiment EEG signals are continually recorded during user performance of each of steps 306 through 312 and after step 312 is completed, recording of the EEG signals is stopped, in some embodiments the EEG signals are only used as part of the performance data for one or more of the tasks, such as the Flanker Arrow task, which is described next. The method 300 proceeds to step 318 and ends.

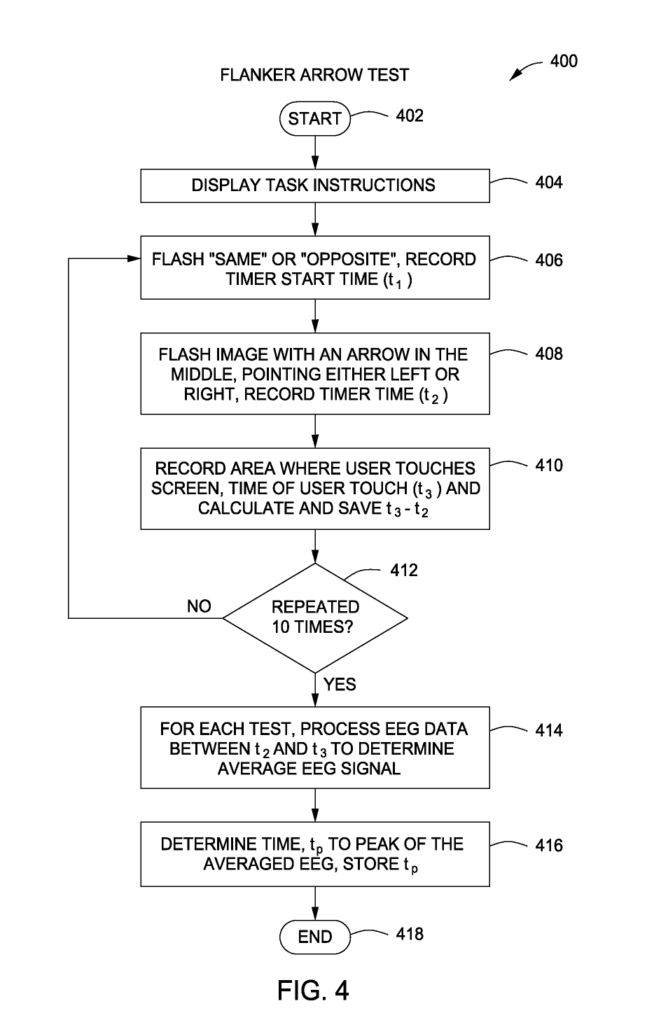

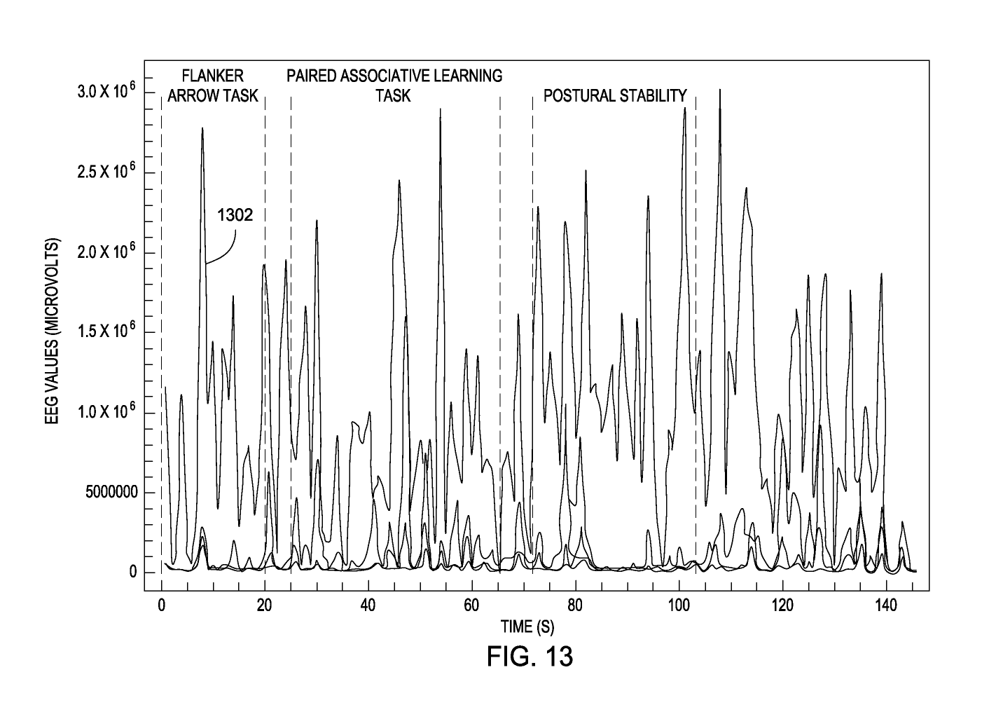

FIG. 4 depicts a flow diagram of a method 400 for administering a QEEG/Flanker Arrow task to the user, as invoked by step 308 of the method 300 of FIG. 3. A unique capability of this embodiment is that it collects and uses EEG data (brain wave signals) in the evaluation of cognitive tasks being performed by the user. The intent of including EEG capabilities is that it can be combined with, for example the Flanker Arrow neurocognitive task, for a measurement of event related potentials. The recorded EEG signals are time locked to the various neurocognitive screening tests, and in the illustrated embodiment the beginning of the Flanker Arrow task coincides with start time t₁. FIG. 13, to be described in detail later, illustrates an exemplary EEG signal acquired from a user during performance of a plurality of the neurocognitive tasks. After being recorded, the stored EEG signals allow calculation by CPU 102, under direction of application 124, of high resolution “P300 waves”. The P300 wave is an event related potential (ERP) elicited by infrequent, task-relevant stimuli, for example the Flanker Arrow task. It is considered to be an endogenous potential as its occurrence links not to the physical attributes of a stimulus but to a person's reaction to the stimulus. In neuroscience, the P300 is thought to reflect processes involved in stimulus evaluation or categorization. As will be described below, the ERP is used in combination with the performance results of the Flanker Arrow task to provide a quantitatively accurate score for the flanker arrow task.

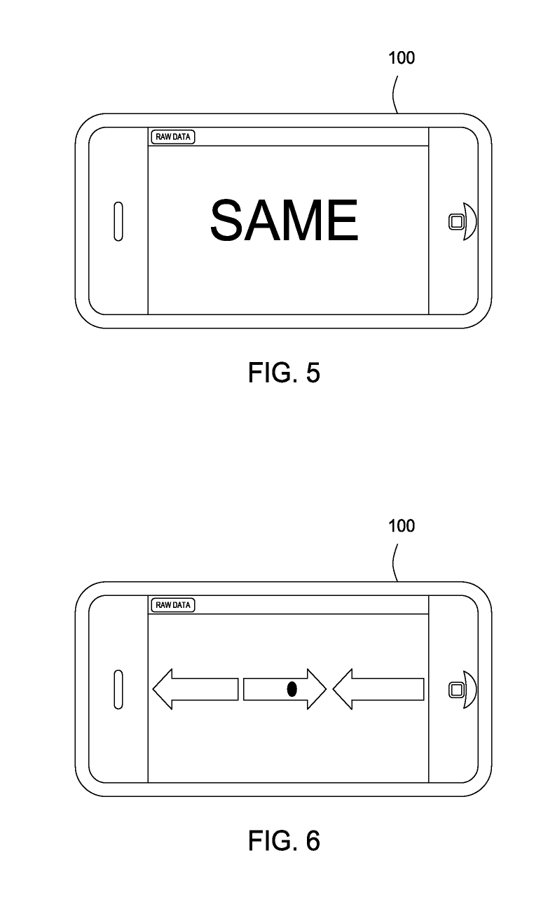

The Flanker Arrow task is a variant of the Eriksen flanker task. The test consists of a cue stimulus followed by a foreperiod and an imperative stimulus. There are two possible cue stimuli, the word “SAME” (the compatible cue) and the word “OPPOSITE” (the incompatible cue). The stimuli appear in random ordering with equal probability. The imperative stimulus is an arrow pointing to the right or to the left, other arrows may flank this arrow or it may stand-alone. The flanking arrows are either congruent, →→→, or incongruent, ←→←, to the middle arrow but they are irrelevant to determination of the correct response. An example of an incongruent imperative stimulus is illustrated in FIG. 6 (described in greater detail below). The correct response is determined by the pairing of the cue and imperative stimuli. If the cue stimulus “SAME” is shown to the user, as illustrated in FIG. 5 (described in greater detail below), the user should press a button displayed on the same side of the touch screen display 108 that the middle arrow points to. Alternatively, if the cue stimulus “OPPOSITE” is shown, the user should press a button displayed on the opposite side of the touch screen display 108 that the middle arrow points to. Response time and accuracy is recorded and stored as data in data portion 126 of memory 106.

The method 400 starts at step 402 and proceeds to step 404. At step 404, the touch screen display 108 of apparatus 100 displays to the user a series of instructions, such as:
“The word SAME or OPPOSITE will flash on the screen. Next, an image will flash. The image will always have an arrow in the middle pointing LEFT or RIGHT. As fast as you can, touch the side of the screen that corresponds to the arrow direction and the word SAME or OPPOSITE. For example, if the arrow is pointing LEFT and the word was OPPOSITE, you should touch the RIGHT of the screen. Another example is if the arrow is pointing LEFT and the word was SAME, you should touch the LEFT of the screen. Always respond as quickly as you can.”

At step 406, the touch screen display 108 will flash a display either the word SAME or OPPOSITE as a cue stimulus to the user in order to begin to administer the Flanker Arrow task, and record the time of the flash as t₁.

At step 408, the touch screen display 108 will flash and image with an arrow in the middle, pointing either left or right so as to display either a congruent stimulus or an incongruent stimulus, and record the time of the flash as t₂.

Referring briefly to FIG. 5, a portion of step 406 is illustrated where the touch screen display 108 of apparatus 100 is shown displaying the word SAME as a cue stimulus to the user in order to administer the Flanker Arrow task. Referring briefly to FIG. 6, a portion of step 408 is illustrated where the touch screen display 108 of apparatus 100 is shown displaying an incongruent imperative stimulus, that is, ←→←.

Referring again to FIG. 4, at step 410 the touch screen display 108 will record the area where the user touches the screen as being either a correct response or an incorrect response, record the user touch as time t₃, and then calculate and save the user's reaction time as t₃−t₂. At step 412, method 400 determines if the Flanker Arrow task has been presented to the user a predetermined amount of times, such as 10 times, and if so proceeds to step 414 and if not repeat steps 406 through 410 until the task has been repeated the predetermined amount of times. At step 414, apparatus 100 processes the EEG data acquired during times t₂−t₃ (shown in detail in FIG. 13) to determine the average EEG signal at these times as acquired from the user during performance of this task. At step 416, apparatus 100 determines the time at tₚ corresponding to the peak of the averaged EEG signal and stores that time as tₚ. The method 400 then proceeds to step 418 and ends.

FIGS. 5 and 6, noted above, illustrate image flashes by the touch screen display 108 of apparatus 100 of the word SAME as a cue stimulus and “←→←”, as an incongruent imperative stimulus, respectively, to the user in order to administer the Flanker Arrow task.

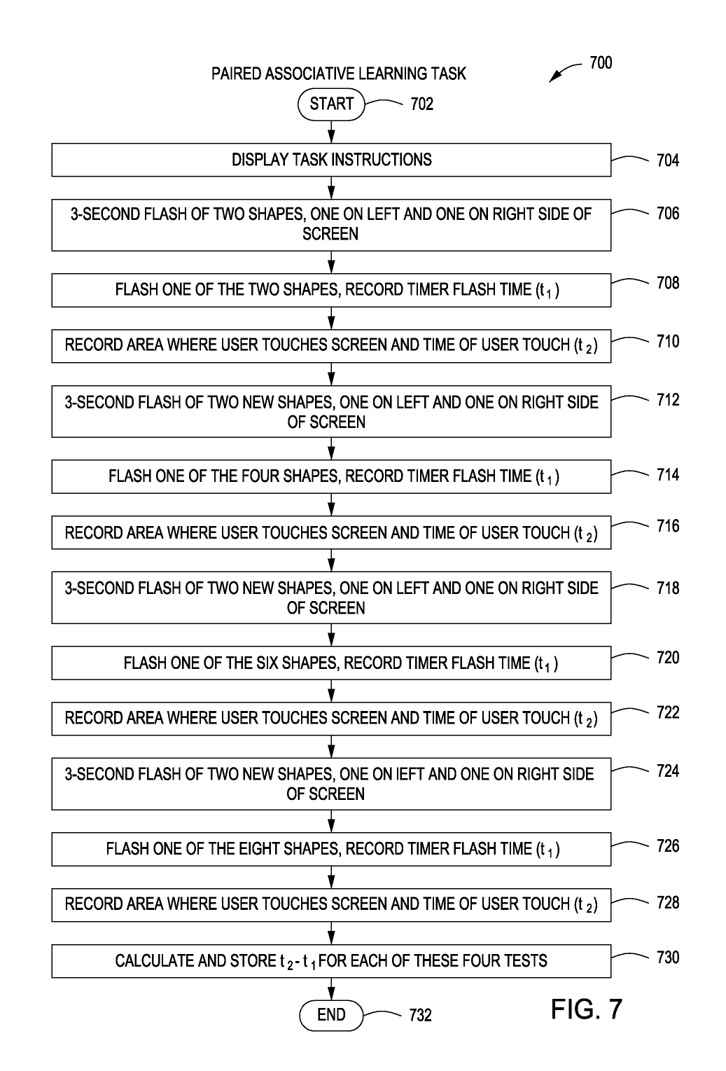

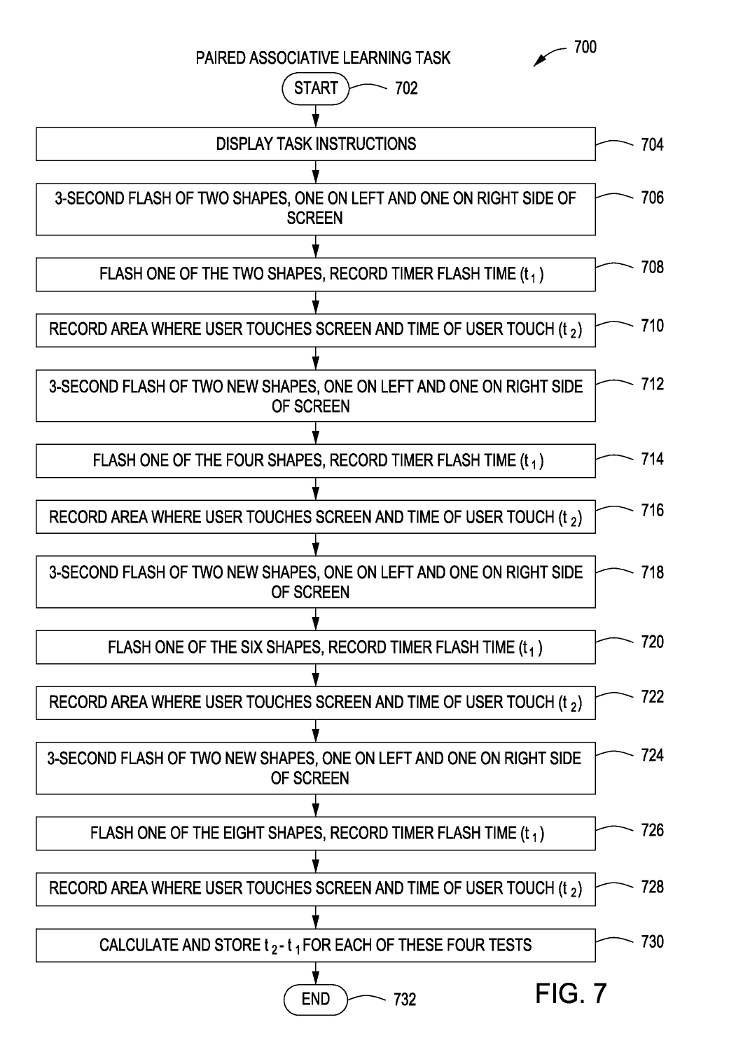

FIG. 7 depicts a flow diagram of a method 700 for administering a Paired Associate Learning (PAL) memory task as invoked by step 310 of the method 300 of FIG. 3. The method 700 starts at step 702 and proceeds to step 704. At step 704, the touch screen display 108 of apparatus 100 displays to the user a series of instructions, such as:
“During this test you will first see two shapes displayed as a pair on the screen side by side. These shapes will be displayed for three seconds. Next, for one second, only one shape will be flashed on the screen. You are to touch the left side of the screen if the flashed shape originally appeared on the left side of the screen. You are to touch the right side of the screen if the flashed shape originally appeared on the right side of the screen. Next, you will see a second pair of two different shapes displayed on the screen side by side. These shapes will also be displayed for three seconds. Then, for one second, only one shape of the two prior pairs of shapes will be flashed on the screen. You are to respond to this one shape depending on which side you first it. This process is repeated until you have had eight shapes to remember. The correctness of your response and your reaction times will be recorded and used in a scoring calculation at the end of the test.”

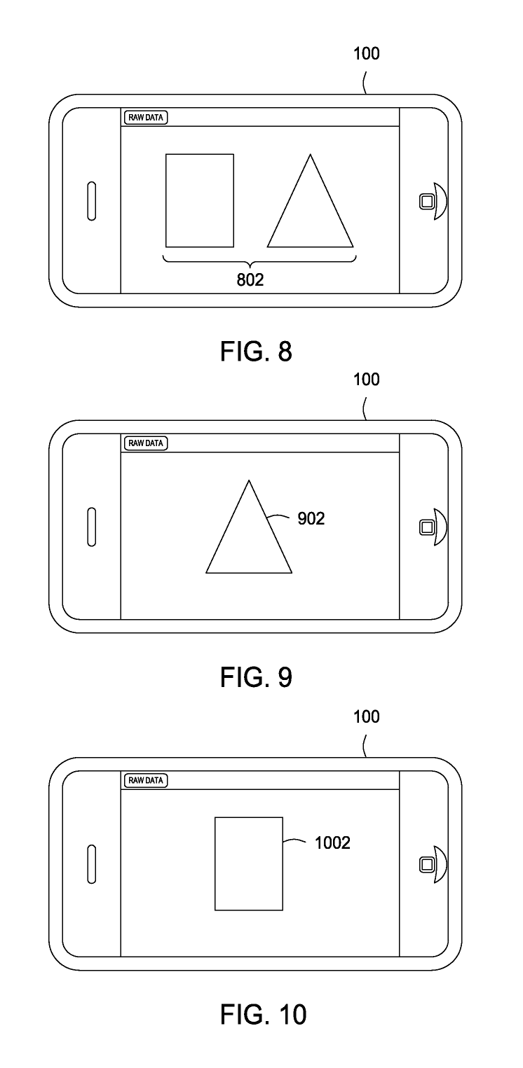

After display of the above instructions to the user, method 700 proceeds to step 706. At step 706, touch screen display 108 will perform a 3-second flash display of a 1st pair of shapes 802 in a side-by-side manner, such as the rectangle and triangle shown in FIG. 8. The method 700 then proceeds to step 708. At step 708 the touch screen display 108 will perform a 1-second flash of one of the two shapes previously shown on the display, record the flash time of the shape as time t₁, and then proceed to step 710. At step 710, the user's response on the touch screen 108 is recorded as being either correct or incorrect in accordance with the users touch on the touch screen display 108 as compared with the original position of that shape on the touch screen display 108. At step 710 the user's response time t₂ is also recorded Illustratively, FIG. 9 depicts a 1-second flash display of a single shape, such as triangle 902, to which the user must respond to by touching the right side of the display to have a correct response in accordance with the position of the paired shapes shown in FIG. 8. FIG. 10 depicts a 1-second flash display of a single shape, such as a rectangle 1002, to which the user must respond to by touching the left side of the display to have a correct response in accordance with the position of the paired shapes shown in FIG. 8. The method 700 then proceeds to step 712.

At step 712, touch screen display 108 will make a 3-second flash display in a side-by-side manner of a 2^{nd} pair of shapes that are different than the 1^{st} pair of shapes previously displayed. The method 700 then proceeds to step 714. At step 714, the touch screen display 108 will perform a 1-second flash of one of the four shapes previously shown on the display, record the flash time of the shape as time t₁, and then proceed to step 716. At step 716, the user's response on the touch screen 108 is recorded as being either correct or incorrect in accordance with the users touch on the touch screen display 108 as compared with the original position of that shape on the touch screen display. At step 716 the user's response time t₂ is also recorded. The method 700 then proceeds to steps 718.

At step 718, touch screen display 108 will make a 3-second flash display in a side-by-side manner of a 3^{rd} pair of shapes that are different than the 1^{st} and 2^{nd} pair of shapes previously displayed. The method 700 then proceeds to step 720. At step 720, the touch screen display 108 will perform a 1-second flash of one of the six shapes previously shown on the display, record the flash time of the shape as time t₁, and then proceed to step 722. At step 722, the user's response on the touch screen 108 is recorded as being either correct or incorrect in accordance with the users touch on the touch screen display 108 as compared with the original position of that shape on the touch screen display, as well as the user's response time t₂. The method 700 then proceeds to steps 724.

At step 724, touch screen display 108 will make a 3-second flash display in a side-by-side manner of a 4^{th} pair of shapes that are different than the 1^{st}, 2^{nd} and 3^{rd} pairs of shapes previously displayed. The method 700 then proceeds to step 726. At step 726, the touch screen display 108 will perform a 1-second flash one of the eight shapes previously shown on the display, record the flash time of the shape as time t₁, and then proceed to step 728. At step 728, the user's response on the touch screen 108 is recorded as being either correct or incorrect in accordance with the users touch on the touch screen display 108 as compared with the original position of that shape on the touch screen display, as well as the user's response time t₂.

The method 700 then proceeds to step 730. At step 730, the uses response time, t₂−t₁ is calculated for each of the responses at steps 710, 716, 722 and 728 and stored as data in data portion 126 of memory 106 of apparatus 100. The method 700 then proceeds to step 732 and ends.

FIGS. 8, 9 and 10, discussed above, illustrate the touch screen display 108 of apparatus 100 displaying shapes during the administration of the PAL memory task where FIG. 8 shows a 3-second side-by-side display flash of a pair of shapes and FIGS. 9 and 10 each show a one-second display flash of a single shape to which the user must respond in accordance with the instructions noted above.

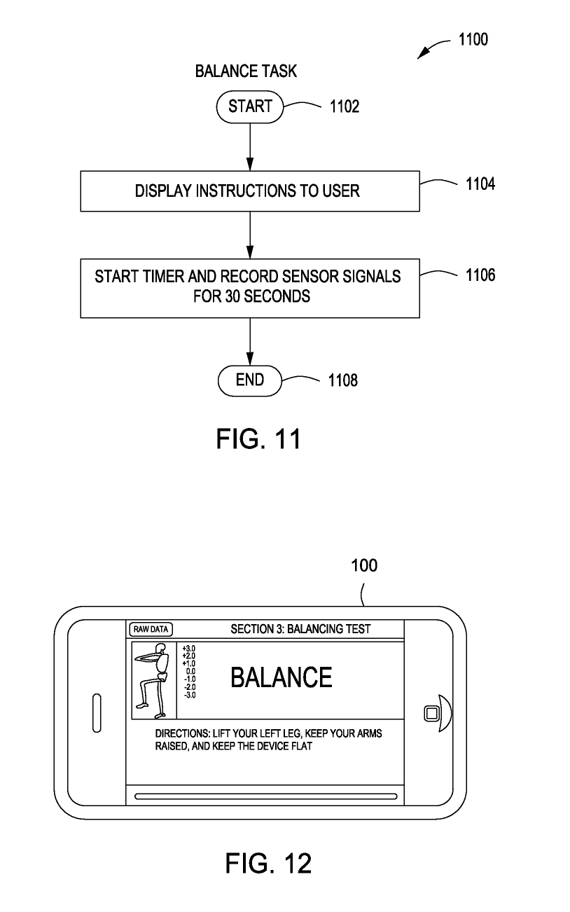

FIG. 11 depicts a flow diagram of a method 1100 for administering a balance task as invoked by step 312 of the method 300 of FIG. 3.

Interesting studies regarding the accuracy of balancing tests have appeared in recent publications. One study claims balancing (i.e., postural stability) tests to be one of the most effective forms of diagnosing brain injury. Balancing in particular involves a complex network of neural connections and centers that are related by peripheral and central feedback mechanisms. A hierarchy integrating the cerebral cortex, cerebellum, basal ganglia, brainstem, and spinal cord is primarily responsible for controlling voluntary movements. In mTBI studies, it has been proposed that communication between sensory systems is lost in a majority of individuals, causing moderate to severe postural instability in the anterior-posterior direction, medial-lateral direction, or both. Typically, postural stability is evaluated in terms of body sway or displacement deviation of the line of action of the person's weight vector through a person's base of support. However, it is difficult to derive and estimate body displacements on a smart phone from on-board accelerometers due to drift and noise of the signals. Note that if the body system is properly constrained, displacement estimates are feasible through basic trigonometry, however the constraints required are unrealistic to be subjected to, for example, a Soldier.

Therefore, in one embodiment an estimate of postural perturbations is used, which estimate is inferred directly from the accelerometers 112 of smart phone apparatus 100 which operate as balance sensors (or stated more generally, as motion sensors) upon invocation of method 1100.

The method 1100 starts at step 1102 and proceeds to step 1104. At step 1104, the touch screen display 108 of apparatus 100 displays to the user, as shown by FIG. 12, a series of instructions, such as:
“Please place the body strap around your torso following the directions on the body strap or as provided by your instructor. Lift your left leg to form a flat surface with your thigh. Your foot should remain flat. Your knee will be raised and the angle of your bend should be approximately 90 degrees. After pressing the start button on the touch screen you will be brought to the testing screen. The testing screen has an image of the balance position you should emulate. Get into that position and press the start button when you are ready. The test will count down from 5 and then start. Please balance on one leg as long as you can for 30 seconds. Even if you lose balance please resume the position and continue balancing until time is up.”

At step 1106, the users balance during a 30 time period is inferred by recording translational accelerations as provided by the accelerometers 112. The method 1100 then proceeds to step 1108 and ends.

FIG. 13, previously described in conjunction with FIG. 4, depicts typical EEG results collected during the multimodal screening tasks administered according to the method 300 of FIG. 3. As described below, at least the EEG results collected during user performance of the Flanker Arrow task are utilized to quantitatively determine the risk index of a brain function impairment.

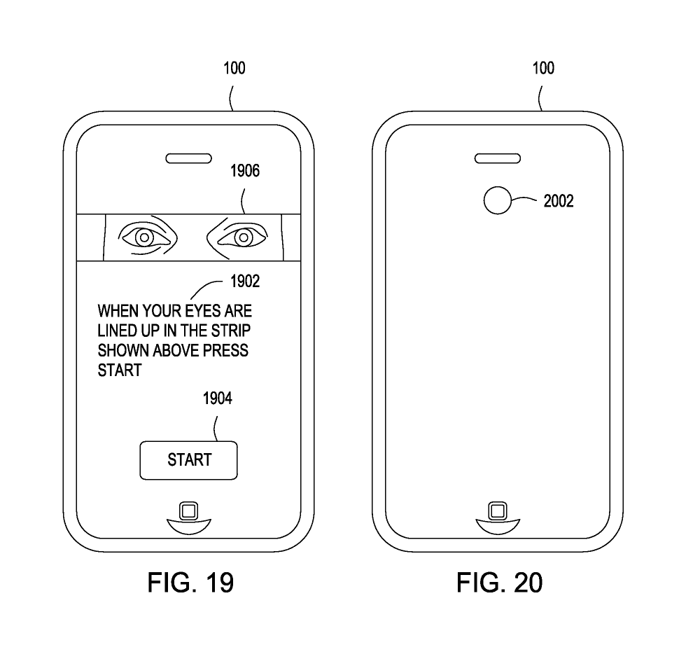

The application software 124 of FIG. 1 computes a risk index of brain function impairment, and more specifically a mTBI risk index screening assessment, as a functional combination of a score determined for each of the tasks performed by the user. In the described embodiment the user performs three tests, 1) QEEG/Flanker Arrow test, 2) the Paired Associate Learning memory test and 3) the postural stability test. In other embodiments additional or alternative tasks could be included as part of this multimodal screening assessment tool, such as an eye tracking task described below in conjunction with FIGS. 19 and 20.

A score for the QEEG/Flanker Arrow test is determined according to reaction time, accuracy, and results from evaluation of the P300 component of the acquired EEG signals. For the Flanker Arrow portion of the test the scoring system is based on the baseline data collected from a study of participants that had no self-reported incidence of mTBI.

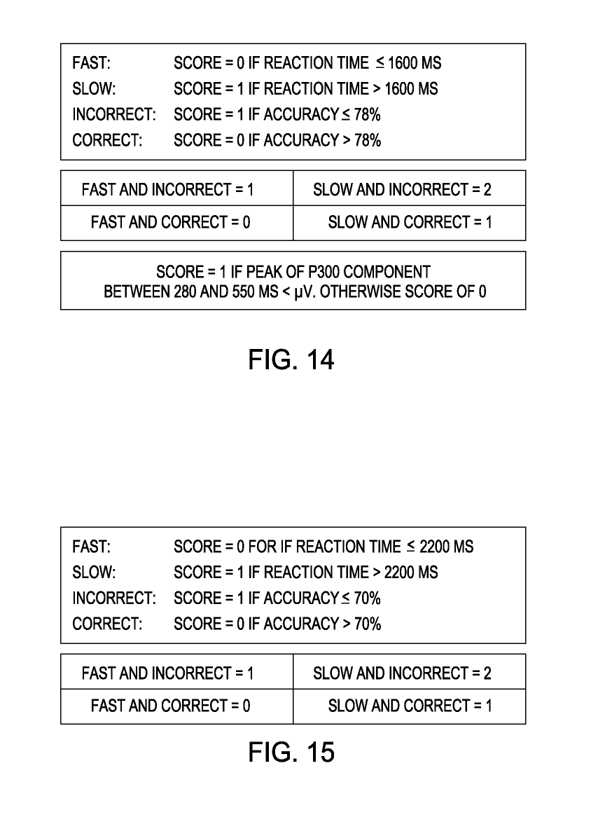

FIG. 14 depicts scoring criteria for the QEEG/Flanker Arrow task of method 400. In the illustrated embodiment the higher the score the more likely a finding of brain function impairment will be found. As shown by the chart of FIG. 14, the user may be “fast” at reacting to the stimulus and “correct” in responding to the arrow direction, in which case they earn the best score (a 0), or the user may “slow” to react and “incorrect”, in which case they earn the worst score (a 2). In order to be considered “fast” the mean reaction time must be less than or equal to 1600 ms, while a “slow” score is given if the mean reaction time is greater than 1600 ms. In one embodiment an incorrect score is given if the mean accuracy of all of the Flanker Arrow tasks is less than or equal to 78% and a correct score is given if mean accuracy is greater than 78%. To score the QEEG portion of the task, for some estimate of the P300 component of the acquired EEG signals, a score of 1 is given if the peak of the P300 component identified as the maximum positive deflection in the time window between 280 and 550 ms from the time of the stimulus is flashed, is less than 2.5 pV (thus, not much brain activity is used to solve the task). It is then assumed that there was no P300 reached. The maximum “worst score for the combined QEEG/Flanker Arrow task of the screening application is 3, with the best score being zero.

FIG. 15 depicts scoring criteria for the Paired Associate Learning memory task of method 700 of FIG. 7. Similar to the QEEG/Flanker Arrow task, scoring this test is based on a study of participants that had no self-reported incidence of mTBI. The user may be “fast” at reacting to the stimulus (shapes displayed on the screen) and “correct” in responding to which side of the screen the shape belongs to, in which case they earn the best score (0), or the user may “slow” to react and “incorrect”, in which case they earn the worst score (2). In order to be considered “fast” the mean reaction time must be less than or equal to 2200 ms, while a “slow” score is considered greater than 2200 ms. For this application an “incorrect “score is given if the mean accuracy of the all the trials is less than or equal to 70% and a correct score is given if mean accuracy is greater than 70%. Although in the illustrated embodiments the acquired EEG signal is not used in scoring of the PAL task, in other embodiments, it may be desirable to use the EEG signal acquired during the users' performance of the PAL task to score this test.

A score for postural stability task is calculated based on the resultant of the de-trended (mean value subtracted) acceleration values in the Cartesian coordinate system, obtained from the accelerometers 112 during the user's performance of the 30 second balancing task of step 1106. Scoring is based according to demerits earned each time the user deviates more than a given amount from a balanced posture. That is, the user starts with a perfect score of 0 and can reduce that score by an amount up to minus 10 based on their balance performance. Artifact-free smart phone based acceleration sensors typically measure 9.81 m/s² (or 1 if normalized by earth's gravity field). This would correspond to holding the phone perfectly still with no movement. When relative movement is experienced and disequilibrium, sway, occurs, the parts of the body accelerate in a direction which deviates from the Earth's gravitational field. Based on a study of participants that had no self-reported incidence of mTBI, resultant accelerations A=\sqrt{aₓ+a_{y}+a_{z}} have been collected and analyzed. For the case of perfect equilibrium, A=1. In accordance with a scoring system of one embodiment described herein, a demerit of 1 is assigned if A becomes greater than 1.2 g or less than 0.8 g (i.e. plus or −20% deviation from being perfectly still) throughout the 30 seconds that the postural stability test is administered. A maximum of 10 demerits can be earned during the 30 seconds, after which the worst value of 10 is used in the final brain function impairment risk index scoring evaluation. The best score is zero.

In one embodiment the risk index of brain function impairment (or of a mTBI), is defined as unity minus the ratio of the sum of the individual test scores to the maximum test score possible, as given by Equation 1. The maximum (or worst) score possible is 15 (3 for the QEEG/Flanker Arrow task, 2 from the Paired Associate Memory task and 10 from the postural stability task).

$$mTBI Risk Index = 1 - ( \frac{{\sum }_{n = 1}^{Number of Tests} Test {Score}_{n}}{{Score}_{m ax}} ) \qquad Equation ( 1 )$$

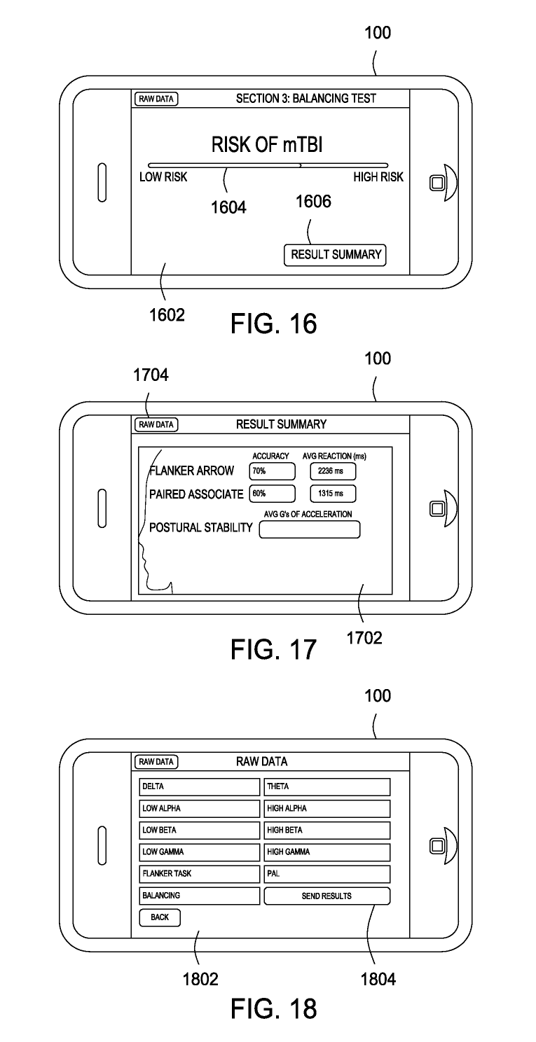

FIG. 16 illustrates a display screen 1602 presented to a user on touch screen display 108 of FIG. 1 upon completion of the above-noted task scoring and determination of the risk index. Display 1602 depicts a simple screen showing the risk index on a sliding scale 1604 having a range from “Low Risk” (corresponding to Equation 1 having a value closer to 1), to “High Risk” (corresponding to Equation 1 having a value closer to 0). If the user touches a “Result Summary” button 1606 of display 1602, a display screen 1702 of FIG. 17 is shown on touch screen display 108 of FIG. 1. Display screen 1702 depicts a “Result Summary” screen illustrating how the user scored on each of the individual tasks of the method of FIG. 3. If the user touches a “Raw Data” button 1704 of display 1702, a display screen 1802 of FIG. 18 is shown on touch screen display 108 of FIG. 1. Display screen 1802 depicts a raw data summary screen illustrating options to display on touch screen display 108 the raw data acquired by each of the individual tasks of the method of FIG. 3, and also gives the user the option, via a button 1804, to send the raw data to another via email.

Many advantages are provided by the described embodiments. Currently, there are limited military or civilian devices to objectively screen for brain function impairment, such as mild traumatic brain injury, and none are portable hand held devices. One value of the described embodiments to the Solider, arises in part from its extreme mobility as well as the quantitative measures that are offered. This extreme mobility allows the screening tests to be completed within a few hours or less of an injury event. Another value of the application is that it can be installed on a widely available device, such as a smart phone, that enables communication of data across networks. This is useful because it enables the medic or corpsman to send raw data, for example EEG data, to a medical expert anywhere in the world.

The described embodiments are also believed to be more accurate than known devices, in that the described embodiments are not subjective in their analysis but objective and quantitative. As with any other computer system, a computer application does what it is programmed to do. Therefore, in terms of measuring core data elements such as acceleration from on-board accelerometers, brain wave signals from an EEG reader and touch inputs and response times from the user as sensed by a touch screen device, the accuracy of the described embodiments is limited only by the upon the hardware it is installed on. As previously noted, in one or more embodiments, the described screening tool is designed to run on most modern smart phones, which typically run on a 1 GigaHertz processor and 512 of Random Access Memory (RAM). For storage, a minimum of 16 Gigabytes of memory is recommended.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. For example, as previously noted, alternative or additional risk assessment tasks can be presented, such as an eye tracking test is illustrated by FIGS. 19 and 20. An eye-tracking portion of the brain injury diagnostic task, being implemented on an APPLE smart phone, would use a View Controller class and a UISubview class within APPLES' Xcode software development kit (SDK) The view controller interacts with the front facing camera on the iPhone 4 with the use of the UIKit. The UIKit allows access to some of the iPhone's hardware, one being the front facing camera. The hardware is interfaced with the use of the UIImagePicker class (also in the Xcode SDK). The image picker handles video interaction by first calling the “is SourceTypeAvailable” class method. This method determines whether or not an onboard camera device is available. The UIImage Picker interface is then told what to display and presents it. The image picker is dismissed using an application delegate object. The UISubview class develops an overlay view called by setting the image picker control properties: UIImagePickerController.cameraOverlayView=UISubview. The user interface has two views, one being an instruction and overlay view and one being an eye tracking view. The test begins by bringing up an instruction view that explains to the user, via instruction 1902, that in this task they are to track with their eyes movement of a green ball that will be displayed on the display. As shown in FIG. 19, the instruction view has a “start” button 1904 linked to an IBAction that calls the overlay view when pressed. The overlay view causes display of a section strip 1906 that shows a view of the test takers eyes taken with the front facing camera of the smart phone, which allows the test taker to line up their eyes in the section strip 1906. The section strip allows for uniform data collection. After the user has their eyes lined up they touch the Start button 1904 to continue. The screen calls another IBAction that blacks out the screen and shows a green ball 2002, as shown in FIG. 20, which ball 2002 the user was previously instructed to track with their eyes. As the user tracks the movement of ball 2002, the smart phone camera takes a video of the users eye movements. The user tracks the ball for a total of 10 seconds; which time can be incremented or decremented as needed through the use of an NSTimer. After the 10 seconds is up, the test will progress to the next question, if desired, and when testing is complete, the video file is saved in the user's photo album and can later be analyzed using eye tracking algorithms. Geotagging, using the GPS circuit portion of a smart phone may be initialized in order to keep track of the location and time a video was taken.

The embodiments described herein were chosen in order to best explain the principles of the present disclosure and its practical applications, to thereby enable others skilled in the art to best utilize the invention and various embodiments with various modifications as may be suited to the particular use contemplated.

Various elements, devices, modules and circuits are described above in associated with their respective functions. These elements, devices, modules and circuits are considered means for performing their respective functions as described herein. While the foregoing is directed to embodiments of the present invention, other and further embodiments of the invention may be devised without departing from the basic scope thereof, and the scope thereof is determined by the claims that follow.

1. Apparatus for administering a plurality of screening tasks to a user for detecting a brain function impairment in the user comprising:
a handheld, portable housing;
a memory, included in the housing, for storing a brain function impairment screening program;
a processor, included in the housing and coupled to the memory, for executing the screening program; and
a display, supported by the housing and responsive to the execution of the screening program, for presenting to the user a plurality of screening tasks to be performed by the user;
wherein the memory stores performance data acquired during the performance of each of the screening tasks by the user and the processor analyzes the acquired data so as to calculate a quantitative risk index indicative of the user having a brain function impairment.

2. The apparatus of claim 1 wherein the display offers the user the option of executing the screening tasks so as to acquire performance data to determine a baseline quantitative risk index or to acquired performance data to determine a quantitative risk index indicative of the user having a brain function impairment.

3. The apparatus of claim 1 wherein the display comprises a touch screen display that presents to the user a plurality of interactive screening tasks to facilitate the acquiring of the performance data during execution of the screening program.

4. The apparatus of claim 1, further comprising a wireless short-range communication module included in the housing and adapted to receive EEG signals transmitted from an EEG sensor that is mounted on the head of a user while the user performs one or more of the presented tasks during execution of the screening program.

5. The apparatus of claim 1, further comprising a motion sensor included in the housing and coupled to the processor to facilitate performance of a balance task performed by the user during execution of the screening program.

6. The apparatus of claim 1, further comprising a camera included in the housing and coupled to the processor to facilitate performance of an eye tracking task performed by the user during execution of the screening program.

7. The apparatus of claim 1, further comprising a long-range wireless communication module included in the housing and adapted to transmit one or more of the quantitative risk index or the acquired data from user performance of one or more screening tasks, to another device.

8. The apparatus of claim 4, where the short-range communication module comprises a Bluetooth transceiver.

9. The apparatus of claim 7, where the long-range wireless communication module comprises a cellular transceiver.

10. The apparatus of claim 1, where the calculated risk index is indicative of the user having an impairment of brain function including at least one of a mild traumatic brain injury, a concussion, a brain disease or a state of inebriation or intoxication.

11. A method for quantitatively detecting a brain function impairment in a user comprising:
presenting a plurality of interactive tasks to a user using a handheld portable device;
acquiring and storing user EEG data representative of a user's brain activity while the user performs one or more of the presented interactive tasks;
acquiring and storing user performance data while the user performs the presented interactive tasks; and
processing the acquired user EEG and performance data so as to determine a quantitative risk index indicative of the user having a brain function impairment.

12. The method of claim 11, where the acquired performance data comprises accuracy and reaction time information relating to the performance of the interactive tasks by the user.

13. The method of claim 11 further comprising presenting the interactive tasks to the user via a touch screen display portion of the handheld portable device.

14. The method of claim 11 further comprising presenting a Flanker Arrow task to the user via a touch screen display portion of the handheld portable device.

15. The method of claim 11, further comprising presenting a Paired Associate Learning task to the user via a touch screen display portion of the handheld portable device.

16. The method of claim 11, further comprising presenting a postural stability task to the user via a screen display and balance sensor portions of the handheld portable device.

17. The method of claim 11, where the interactive tasks presented to the user are intended to task different areas of the users' brain.

18. The method of claim 11, further comprising presenting an eye-tracking task to the user via a display and camera portions of the handheld portable device.

19. The method of claim 11, further comprising determining the risk index as a normalized result of a functional combination of the users' acquired and stored EEG data and performance data.

20. The method of claim 19, further comprising determining a Test Score by evaluating the performance data for each interactive task performed by the user.

21. The method of claim 19 further comprising determining the Risk Index according to the following equation:
$$Risk Index = 1 - ( \frac{{\sum }_{n = 1}^{Number of Tests} Test {Score}_{n}}{{Score}_{m ax}} )$$

22. The method of claim 11, further comprising determining the risk index to be indicative of the user having an impairment of brain function that includes at least one of a mild traumatic brain injury, a concussion, a brain disease or a state of inebriation or intoxication.
